(19) United States
(12) Patent Application Publication
(10) Pub. No.: US 2023/0402149 A1
(43) Pub. Date: Dec. 14, 2023

(54) SWITCHING AND CUSTOMIZATION OF GLUCOSE PREDICTION MODELS IN MEDICAMENT DELIVERY DEVICES

(71) Applicant: INSULET CORPORATION, Acton, MA (US)

(72) Inventors: Joon Bok LEE, Acton, MA (US); William WHITELEY, Knoxville, TN (US); Saeed SALAVATI, Sugar Land, TX (US); Yibin ZHENG, Hartland, WI (US); Mert SEVIL, Manchester, NH (US); Jason O’CONNOR, Acton, MA (US)

(21) Appl. No.: 18/199,638

(22) Filed: May 19, 2023

Related U.S. Application Data
(60) Provisional application No. 63/343,739, filed on May 19, 2022.

(51) Int. Cl. G16H 20/17; A61B 5/145; A61B 5/00

(52) U.S. Cl. CPC G16H 20/17; A61B 5/7275; A61B 5/14532

(57) ABSTRACT

Exemplary embodiments may provide for the switching of glucose prediction models responsive to certain conditions. For example, glucose prediction models may be switched responsive to a detected crashing glucose level condition. Exemplary embodiments also may dynamically customize parameters, such as coefficient values, of the glucose prediction model to a user. The exemplary embodiments may customize the glucose prediction model based on the history of glucose levels of the user and the history of insulin deliveries to the user. The exemplary embodiments may determine a set of parameters that provides an improved fit of the parameters to the history of glucose levels and insulin deliveries of the user. The improved fit parameters may be used to adapt the parameter set to the most recent run.

### RELATED APPLICATIONS

This application claims the benefit of U.S. Provisional Patent Application No. 63/343,739, filed May 19, 2022, the entire contents of which are incorporated herein by reference in their entirety.

### BACKGROUND

Some conventional automated insulin delivery (AID) systems rely on glucose prediction models that predict the future glucose levels of a user. These glucose prediction models may predict future glucose level values of the user based at least in part on the most recent glucose level values of the user and insulin deliveries to the user that may still have an effect on glucose level values of the user. The predicted future glucose level values of the user may be used in controlling basal insulin deliveries to the user. As such, it is desirable to have the predicted future glucose level values be accurate in order for the AID systems to deliver suitable insulin doses to the user in basal insulin deliveries.

One complication with some conventional glucose prediction models is that the glucose prediction models are slow to respond to rapid decreases in glucose level values of the user. As a result, the future glucose level values that are predicted are too high. Hence, the AID systems that deploy such conventional glucose prediction models may continue to deliver insulin to a user even though the glucose level of the user is falling rapidly. The extra insulin delivery magnifies the plunge in glucose level values of the user.

Another challenge with conventional glucose prediction models used in conventional AID systems is that the conventional glucose prediction models may not be well suited for many users. Generally, the conventional glucose prediction models are formulated by assessing average characteristics for a population. Unfortunately, as a result, for many users the accuracy of the glucose prediction models is poor. This may in large part reflect varying insulin sensitivities among users. The conventional glucose prediction models tend to be conservative in their responsiveness to avoid under-delivery or over-delivery of insulin to users.

### SUMMARY

In accordance with an inventive facet, a medicament delivery device for delivering insulin to a user includes a non-transitory computer-readable storage medium storing computer programming instructions and a processor configured for executing the computer programming instructions. Executing the computer programming instructions causes the processor to predict a first future glucose level of the user using a first model that predicts the future glucose level based on glucose level history of the user and insulin deliveries to the user and to detect a crashing glucose level condition of the user. Executing the computer programming instructions also causes the processor, in response to the detecting of the crashing glucose level condition, to switch to a second model to predict a next future glucose level of the user, wherein the second model is more conservative than the first model such that the second model predicts lower future glucose levels for the user than the first model when glucose levels of the user are decreasing.

The processor may detect the crashing glucose level condition by determining differences between successive pairs of glucose level readings of a sequence of most recent glucose level readings of the user that extend from an oldest glucose level reading in the sequence to a most recent glucose level reading in the sequence and comparing each of the differences to a threshold. The processor may detect the crashing glucose level condition by comparing the differences to each other to determine if the differences between the successive pairs of glucose level readings of the user become more negative as the differences range from an oldest glucose level reading pair to a most recent glucose level reading pair. The processor may detect a crashing glucose level condition of the user when each of the differences is more negative than the threshold, each of the differences is negative, and the differences between the successive pairs of glucose level readings of the user become more negative as the differences range from the oldest glucose level reading pair to the most recent glucose level reading pair.

The detecting of a crashing glucose level condition may entail comparing Insulin on Board (IOB) of the user to a threshold. The processor may detect the crashing glucose level condition by looking for glucose level readings of the user dropping at an accelerating rate. In response to the detecting of the crashing glucose level condition, the processor may decrease an upper bound of an amount of insulin that may be delivered by the medicament delivery device to the user over a time period. The computer programming instructions when executed may further cause the processor to determine a basal insulin delivery dose for the user based on the predicted next future glucose level of the user that was predicted using the second model. The medicament delivery device may include a cannula and/or needle for delivering the medicament to the user, and the computer programming instructions when executed by the processor may further cause the processor to initiate delivery of the basal insulin delivery dose to the user via the needle and/or cannula.

In accordance with another inventive facet, a medicament delivery device for delivering insulin to a user may include a non-transitory computer-readable storage medium storing computer programming instructions and a processor configured for executing the computer programming instructions. Executing the computer programming instructions may cause the processor to access a history of insulin delivery doses and glucose level values of the user. Based on the history, the processor may determine an improved fit of parameters for a glucose prediction model from the history relative to current parameters of the glucose prediction model, wherein the glucose prediction model determines predicted future glucose level values for the user from past glucose level values and past insulin delivery doses in the history, such that glucose prediction model using the improved fit parameters more accurately predicts future glucose level values than the glucose prediction model using the current parameters to predict future glucose level values when compared to predicting glucose level values in the history of glucose level values of the user. Executing the computer programming instructions may further entail adapting the parameters based on the improved fit of parameters to produce adapted parameters and may cause the processor to use the glucose prediction model with the adapted parameters to predict at least one a next future glucose level value of the user.

In determining the improved fit of parameters for the glucose prediction model, the glucose prediction model may determine a best fit of parameters for the glucose prediction model. The improved fit may be the best fit of parameters for the glucose prediction model. The processor may use a genetic algorithm to determine the improved fit of the parameters. The medicament delivery device may have operational cycles, may receive at least one glucose level reading each operational cycle, and may deliver a basal insulin dose to the user each operational cycle. The processor in determining the improved fit of parameters may examine over multiple operational cycles of the medicament delivery device. A maximum deviation of a parameter relative to a population average for parameter candidates may be established and at least one of the parameters in the improved fit of parameters may be subject to the maximum deviation. Parameters in the improved fit of parameters may be required to conform with a stability constraint. Glucose level values in the history of glucose level values that are affected by disturbances may be removed from consideration in determining the improved fit of parameters.

In accordance with an additional inventive facet, a medicament delivery device for delivering medicament to a user includes a non-transitory computer-readable storage medium storing computer programming instructions and a processor that is configured for executing the computer programming instructions. Executing the computer programming instructions may cause the processor to use a current model to predict a response in analyte levels of the user to delivering a dose of medicament. Executing the computer programming instruction may further cause the processor to, responsive to detecting a condition, swap the current model or parameters for another model or parameters, or to update the current model based on new analyte level data. In addition, executing the computer programming instructions may cause the processor to use the other model or the updated current model to choose a medicament dose for the user.

The detected condition may be rapidly changing analyte level data. The modification may be to halt delivery of the medicament or may be modifying parameters of the model for medicament dose calculation.

### BRIEF DESCRIPTION OF THE DRAWINGS

FIG. 1 depicts a block diagram of an illustrative medicament delivery system that is suitable for delivering a medicament to a user in accordance with the exemplary embodiments.

FIG. 2 depicts a flowchart of illustrative steps that may be performed in exemplary embodiments in using the glucose prediction model.

FIG. 3 depicts a flowchart of illustrative steps that may be performed in exemplary embodiments in realizing a switch between glucose prediction models.

FIG. 4 depicts a flowchart of illustrative steps that may be performed in exemplary embodiments regarding switching back from a second model to a first model.

FIG. 5 depicts a flowchart of illustrative steps that may be performed in exemplary embodiments in detecting a crashing glucose level condition.

FIG. 6 depicts a flowchart of illustrative steps that may be performed in exemplary embodiments regarding modifying insulin delivery constraints.

FIG. 7 depicts a flowchart of steps that may be performed in exemplary embodiments in customizing the parameters of the glucose prediction model for the user.

FIG. 8 depicts a flowchart depicting illustrative steps that may be performed in an illustrative approach to generate an improved fit parameter set in accordance with exemplary embodiments.

FIG. 9 depicts a table of illustrative values for a generation of parameter sets.

FIG. 10 depicts a flowchart of illustrative steps that may be performed in exemplary embodiments to find an improved fit of parameters across multiple cycles.

FIG. 11 depicts a flowchart of illustrative steps that may be performed in exemplary embodiments to constrain parameters.

FIG. 12 depicts a flowchart of illustrative steps that may be applied in exemplary embodiments relating to the stability constraint.

FIG. 13 depicts a flowchart of illustrative steps that may be performed in exemplary embodiments to remove the effect of a disturbance.

FIG. 14 depicts a flowchart of illustrative steps that may be performed in exemplary embodiments to remove glucose level values affected by a disturbance.

FIG. 15 depicts a flowchart of illustrative steps that may be performed to adapt parameters.

FIG. 16 depicts a flowchart of illustrative steps that may be performed in exemplary embodiments to update parameters using a Kalman filter.

### DETAILED DESCRIPTION

Exemplary embodiments may provide for the switching of glucose prediction models responsive to certain conditions. For example, glucose prediction models may be switched responsive to a detected crashing glucose level condition. Conventional glucose prediction models tend to keep the same fixed model parameters even when there is an accelerating rate of glucose level decrease for a period, and hence delivering insulin for a period after glucose levels of a user are crashing. The continued delivery of insulin under such a condition may only magnify the problem posed by the crashing glucose level condition. The exemplary embodiments may avoid this problem of continued insulin delivery by switching to a model that is more aggressive in terminating or decreasing insulin delivery responsive to detection of the crashing glucose level condition of the user. The glucose prediction model of exemplary embodiments may be configured to predict glucose levels of the user more accurately when blood glucose levels are decreasing at an accelerating rate than conventional glucose prediction models. Hence, insulin deliveries may be decreased or halted more quickly under such a condition.

The exemplary embodiments also may modify insulin delivery constraints upon detecting the crashing glucose level condition. For instance, exemplary embodiments may reduce a one-time upper bound constraint that limits the basal insulin delivery rate by the medicament delivery device when a crashing glucose level condition is detected. The exemplary embodiments further may reduce an integral constraint that constrains how much basal insulin may be delivered over a predetermined period, such as a few hours, when a crashing glucose level condition is detected.

Exemplary embodiments may customize parameters, such as coefficient values, of the glucose prediction model to a user. The exemplary embodiments may customize the glucose prediction model based on the history of glucose levels of the user and the history of insulin deliveries to the user. The exemplary embodiments may determine a set of parameters that provides an improved fit of the parameters to the history of glucose levels of the user relative to a run of recent glucose level values. The improved fit may be a best fit relative to the recent glucose level values in some exemplary embodiments. Strategies, such as genetic algorithms and other parameter fitting strategies, may be used in the exemplary embodiments. The improved fit parameters are then used in adapting the current parameters to the recent glucose level values.

Although the discussion will focus on the case where the medicament delivery device is an insulin delivery device and the measured analyte is glucose level of a user, it should be appreciated the systems, methods and storage media described herein may also be used in delivery of other medicaments than insulin or insulin alone and may rely concern analyte levels other than glucose such as ketone levels of a user. Exemplary medicaments that may be used include insulin, glucagon-like peptide-1 receptor agonist (GLP-1), glucose-dependent insulinotropic polypeptide (GIP), or other hormones, and/or combinations of medicaments, such as two or more of insulin, GLP-1, and GIP, or other like hormones or weight-loss drugs.

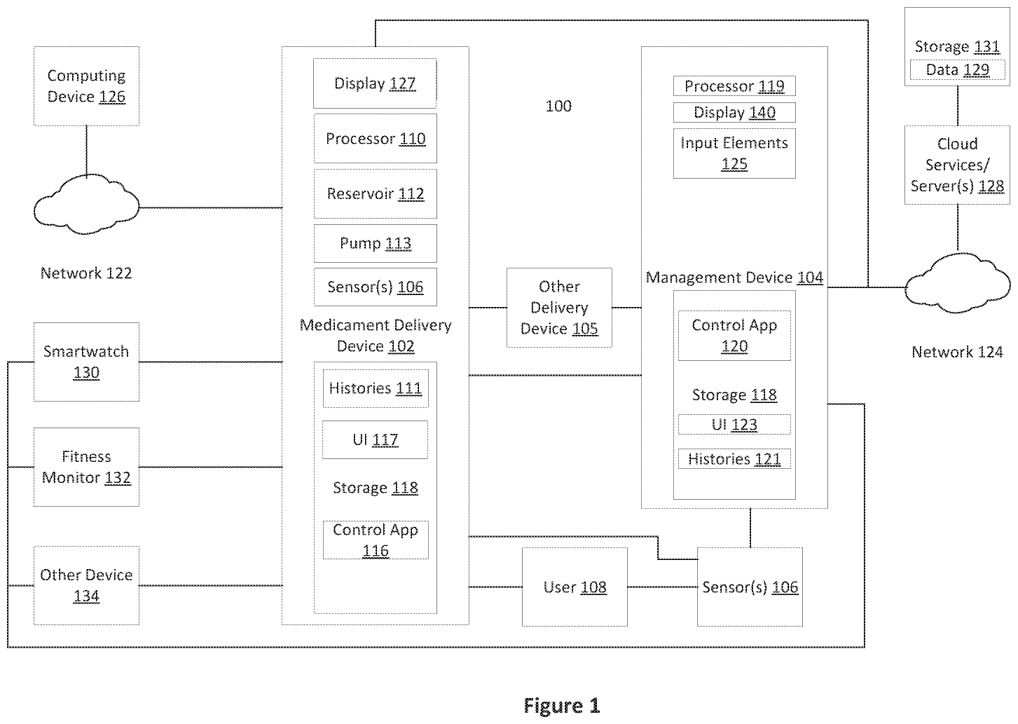

FIG. 1 depicts a block diagram of an illustrative medicament delivery system 100 that is suitable for delivering a medicament, such as insulin, to a user 108 in accordance with the exemplary embodiments. The medicament delivery system 100 may include a medicament delivery device 102. The medicament delivery device 102 may be a wearable device that is worn on the body of the user 108 or carried by the user. The medicament delivery device 102 may be directly coupled to a user (e.g., directly attached to a body part and/or skin of the user 108 via an adhesive or the like) with no tubes and an infusion location directly under the medicament delivery device 102, or carried by the user (e.g., on a belt or in a pocket) with the medicament delivery device 102 connected to an infusion site where the medicament is injected using a needle and/or cannula. A surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries or the like. The processor 110 also may execute computer programming instructions stored in the storage 114 for a user interface (UI) 117 that may include one or more display screens shown on display 127. The display 127 may display information to the user 108 and, in some instances, may receive input from the user 108, such as when the display 127 is a touchscreen.

The control application 116 may control delivery of the medicament to the user 108 per a control approach like that described herein. The control application may use a glucose prediction model as described below for predicting future glucose levels of the user 108. The storage 114 may hold histories 111 for a user, such as a history of basal deliveries, a history of bolus deliveries, and/or other histories, such as a meal event history, exercise event history, glucose level history, other analyte level history, and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include a tray or cradle and/or one or more housings for housing its various components including a pump 113, a power source (not shown), and a reservoir 112 for storing medicament for delivery to the user 108. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112), and may include a conduit to a separate infusion site. The medicament delivery device 102 may have operational cycles, such as every 5 minutes, in which basal doses of medicament are calculated and delivered as needed. These steps are repeated for each cycle.

There may be one or more communications links with one or more devices physically separated from the medicament delivery device 102 including, for example, a management device 104 of the user and/or a caregiver of the user, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN), a cellular network, a Wi-Fi network, a near field communication network, or a combination thereof. A computing device 126 may be interfaced with the network 122, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions, physical attributes, or analyte levels of the user 108. The sensor(s) 106 may be physically separate from the medicament delivery device 102 or may be an integrated component thereof. The sensor(s) 106 may include, for example, glucose monitors, such as continuous glucose monitors (CGM's) and/or non-invasive glucose monitors. The sensor(s) 106 may include ketone sensors, other analyte sensors, heart rate monitors, breathing rate monitors, motion sensors, temperature sensors, perspiration sensors, blood pressure sensors, alcohol sensors, or the like. Some sensors 106 may also detect characteristics of components of the medicament delivery device 102. For instance, the sensors 106 in the medicament delivery device may include voltage sensors, current sensors, temperature sensors and the like.

The medicament delivery system 100 may or may not also include a management device 104. In some embodiments, no management device is needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensor(s) 106. The management device 104 may be any portable electronic device including, for example, a dedicated device, a smartphone, a smartwatch, or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. Storage 118 may also be operable to store historical information such as medicament delivery information, analyte level information, user input information, output information, or other historical information. The control application 120 may be responsible for controlling the medicament delivery device 102, such as by controlling the automated medicament delivery (ADD) (or, for example, automated insulin delivery (AID)) of medicament to the user 108. The storage 118 may store the control application 120, histories 121 like those described above for the medicament delivery device 102, and other data and/or programs.

A display 140, such as a touchscreen, may be provided for displaying information. The display 140 may display user interface (UI) 123. The display 140 also may be used to receive input, such as when it is a touchscreen. The management device 104 may further include input elements 125, such as a keyboard, button, knobs, or the like, for receiving input form the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128.

Other devices, like smartwatch 130, fitness monitor 132 and device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user, such as to request a change or pause in dosage, or to request, initiate, or confirm delivery of a bolus of medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data. Another delivery device 105, such as a medicament delivery pen (such as an insulin pen), may be accounted for (e.g., in determining JOB) or may be provided for also delivering medicament to the user 108.

The functionality described herein for the exemplary embodiments may be under the control of or performed by the control application 116 of the medicament delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another wearable device 134.

In the closed loop mode, the control application 116, 120 determines the medicament delivery amount for the user 108 on an ongoing basis based on a feedback loop. For a medicament delivery device that uses insulin, for example, the aim of the closed loop mode is to have the user's glucose level at a target glucose level or within a target glucose range.

In some embodiments, the medicament delivery device 102 need not deliver one medicament alone. Instead, the medicament delivery device 102 may one medicament, such as insulin, for lowering glucose levels of the user 108 and also deliver another medicament, such as glucagon, for raising glucose levels of the user 108. As explained above, the medicament delivery device 102 may deliver other medicaments in lieu of insulin, such as a glucagon-like peptide (GLP)-1 receptor agonist medicament for lowering glucose or slowing gastric emptying, thereby delaying spikes in glucose after a meal. In other embodiments, the medicament delivery device 102 may deliver pramlintide, or other medicaments that may substitute for insulin. In other embodiments, the medicament delivery device 102 may deliver concentrated insulin. In some embodiments, the medicament or medicament delivered by the medicament delivery device may be a coformulation of two or more of those medicaments identified above. In a preferred embodiment, the medicament delivery device delivers insulin; accordingly, reference will be made throughout this application to insulin and an insulin delivery device, but one of ordinary skill in the art would understand that medicaments other than insulin can be delivered in lieu of or in addition to insulin.

Exemplary embodiments may use analyte prediction models to predict future analyte levels for a user. For example, the analyte level being predicted may be a future glucose level of a user and the medicament may be insulin in some exemplary embodiments. The glucose prediction model may predict a future glucose level for the user as:

G_{Δ}(k)=b₁G_{Δ}(k−1)+b₂G_{Δ}(k−2)+b₃G_{Δ}(k−3)−KI_{Δ}(k−3) (Equation 1)

where G_{Δ}(k) is a predicted glucose level value for cycle k expressed as a delta relative to the setpoint, G_{Δ}(k−1), G_{Δ}(k−2), and G_{Δ}(k−3) are the predicted glucose level values for cycles k−1, k−2, and k−3 expressed as deltas relative to the setpoint, respectively, b₁, b₂, b₃ are pre-determined prediction coefficients, K is a weight coefficient, and I_{Δ}(k−3) is the insulin dose delivered at cycle k−3 expressed as a difference relative to the standard basal delivery dose.

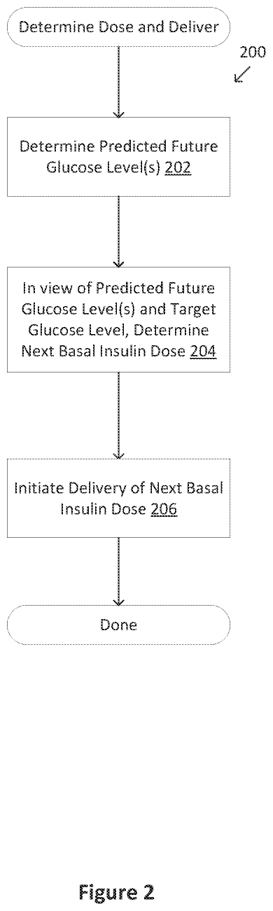

FIG. 2 depicts a flowchart 200 of illustrative steps that may be performed in exemplary embodiments in using the glucose prediction model. At 202, the glucose prediction model may be used by the control application 116 or 120 to determine one or more predicted future glucose level values for the user from the glucose level values and insulin delivery dose information in the histories 111 and/or 121. The glucose prediction model may predict a glucose value for only a single future operational cycle of the medicament delivery device 102 or for multiple future operational cycles. At 204, in view of the predicted glucose level(s) and the target glucose level (e.g., the setpoint) the next basal insulin delivery dose may be determined. A cost function with a glucose cost component and an insulin cost component may be used in determining the basal insulin delivery dose. The insulin dose candidate with lowest cost may be chosen as the next basal insulin delivery dose by the control application 116 or 120. A suitable cost function is:

J(t)=Q·Σᵢ₌₁^{M}(CGM(i)−target(i))²+R·Σᵢ₌₁^{N}(Iₚ(i)−I_{b}(i))² (Equation 2)

where J(t) is the cost of an insulin dose for cycle t, target(i) is a target glucose level for the user for cycle i, CGM(i) is the expected glucose level of the user at cycle i, M is the last cycle in a future time horizon over which the glucose cost is determined, Q is the weight coefficient for the glucose cost component, Iₚ(i) is the expected insulin dose at cycle i, I_{b}(i) is the expected ideal basal dose for cycle i (which may be based on a user's total daily insulin (TDI) value divided by 24 divided by 2), N is the cycle number of the last cycle over which the insulin cost is determined, and R is the insulin cost weight coefficient. QΣᵢ₌₁^{M}(CGM(i)−target(i))² is the glucose cost component of the cost function and R·Σᵢ₌₁^{N}(Iₚ(i)−I_{b}(i))² is the insulin cost component of the cost function. As can be seen from the above equation, the predicted future glucose level values figure into the glucose cost component of the above cost function. Lastly, at 206, delivery of the determined basal insulin dose may be initiated by the medicament delivery device 102 to the user 108.

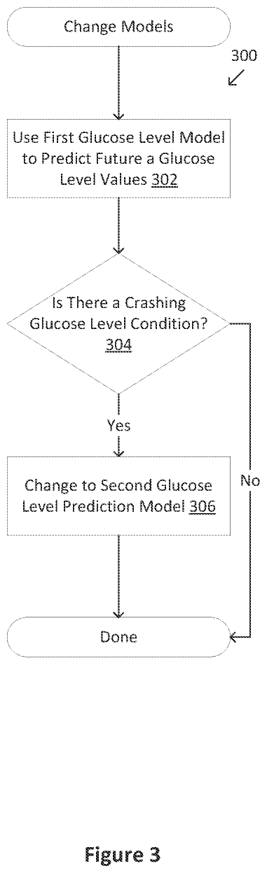

As mentioned above, exemplary embodiments may switch glucose prediction models responsive to some conditions. One such condition is a crashing glucose level condition. The exemplary embodiments may change from one glucose prediction model to another responsive to detecting one or more conditions. For example, the same glucose prediction function (see Equation 1) may be used by the models but the coefficients (b₁, b₂, and b₃) may change. The change in coefficients is termed herein as a change in the model. FIG. 3 depicts a flowchart 300 of illustrative steps that may be performed in exemplary embodiments in realizing a switch in glucose prediction models. Initially, at 302, a first glucose prediction model may be used to predict future glucose level values. This first model may be well suited for most operating conditions. However, the first model may not be well suited for certain conditions. One of those conditions may be a crashing glucose level condition, where the glucose level of the user is falling at an accelerating rate over a period. At 304, a check of whether there is a crashing glucose level condition may be performed. If not, there is no need for a change in the model so the process is done. If there is a crashing glucose level condition, at 306, a change may be made in the glucose prediction model to a second model that is better suited for responding to a the crashing glucose level condition. The coefficients used in the second model may show or yield a greater drop in predicted glucose levels more quickly than the first model during a crashing glucose level condition and thus may react to the crashing glucose level condition more quickly than the first model. The first model may be said to be more aggressive as it is more likely to deliver insulin more easily than the second model and may deliver larger doses than the second model. Hence, the second model may be characterized as less aggressive or more conservative.

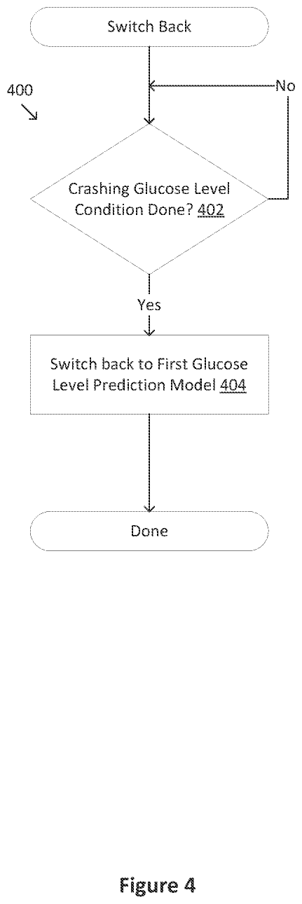

In exemplary embodiments, the second glucose prediction model may continue to be used until the crashing glucose level condition abates. FIG. 4 depicts a flowchart 400 of illustrative steps that may be performed in exemplary embodiments regarding switching back from the second model to the first model. At 402, a check may be made whether the crashing glucose level condition is still occurring or not. This may entail checking whether the conditions that were tested (such as described below) to identify whether a crashing glucose level condition is still met or not. If not, no switch back occurs. If the conditions that were tested indicate that the crashing glucose level condition is done, at 404, a switch back to the first glucose prediction model may be performed so that the first glucose level prediction model is used.

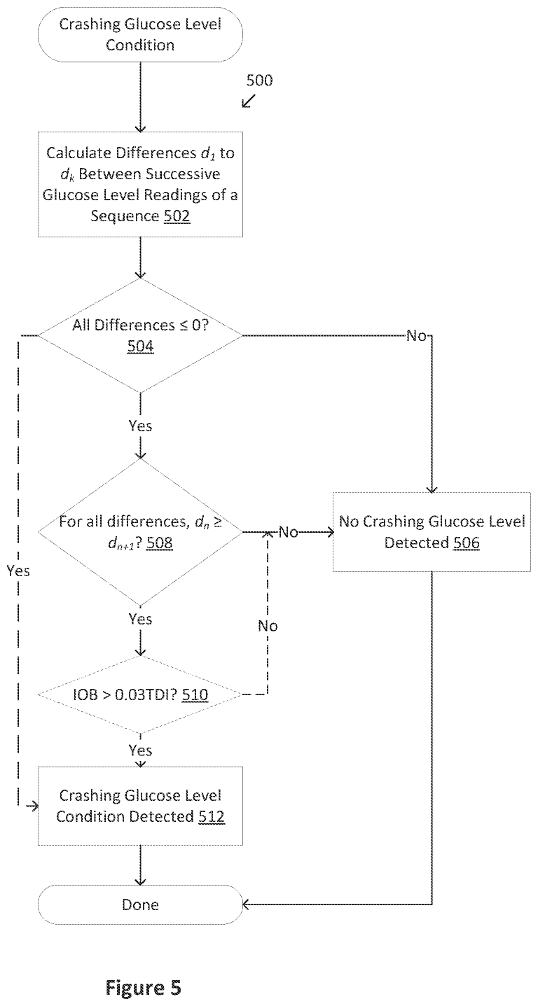

The detection of a crashing glucose level condition may be determined in a variety of ways. FIG. 5 depicts a flowchart 500 of illustrative steps that may be performed in one of these ways of detecting a crashing glucose level condition. In this example, for a crashing glucose level condition to be detected, the differences between successive predicted glucose levels in a sequence must indicate that the glucose level of the user has been decreasing at an accelerating rate over a period. Hence, at 502, differences between successive predicted glucose level readings of the user may be determined. The sequence may contain most recent predicted glucose level readings of the user. In some examples, the three most recent predicted glucose level values from the histories 111 and/or 121 may be used. In that case, the differences may be calculated as:

d₃=G(k−3)−G(k−2), (Equation 3)

d₂=G(k−2)−G(k−1), (Equation 4)

d₁=G(k−1)−G(k) (Equation 5)

where G(k) is the predicted glucose level value for cycle k, G(k−1) is the predicted glucose level of the user for cycle k−1, G(k−2) is the predicted glucose level of the user for cycle k−2, G(k−3) is the predicted glucose level of the user for cycle k−3 and d₁, d₂, and d₃ are differences between successive glucose level values.

At 504, a check may be made as to whether all of the differences are less than or equal to zero. The glucose level is not likely to be crashing if a difference is not negative. Thus, if not all of the differences for the sequence of successive predicted glucose level values are less than or equal to zero, at 506, no crashing glucose level condition may be detected. If all of the differences are less than or equal to zero, a crashing glucose level condition may be detected. Additionally or alternatively, at 508, a further check may be made whether the rate of decrease in predicted glucose level values is accelerating by checking for all of the differences other than the last difference being more negative than the predecessor difference. This may be expressed as checking whether dₙ is greater than equal to dₙ₊₁ for all but the last difference in the sequence. If not, no crashing glucose level condition may be detected at 506. If so, at 512, the crashing glucose level condition may be considered as being detected. In some embodiments, the process may also include an optional check relating to IOB at 510 (shown in phantom form to indicate that it is optional). Specifically, a check may be made whether the IOB of the user is greater than 3% of total daily insulin (i.e., the average sum of basal insulin deliveries and bolus insulin deliveries per day). The rationale for this check at 510 is that if the user has little IOB (e.g. less than 3% of TDI), the user is at much less risk of hypoglycemia if more insulin is delivered to the user and thus quickly decreasing the insulin delivery responsive to a crashing glucose level condition matters less and there is less need to switch glucose prediction models. If the user has IOB greater than 3% TDI, then a crashing glucose level condition may be declared as detected at 512. Otherwise, no crashing glucose level condition may be detected at 506. Values other than 3% may be used.

It should be appreciated that a crashing glucose level condition may be determined in other ways as well.

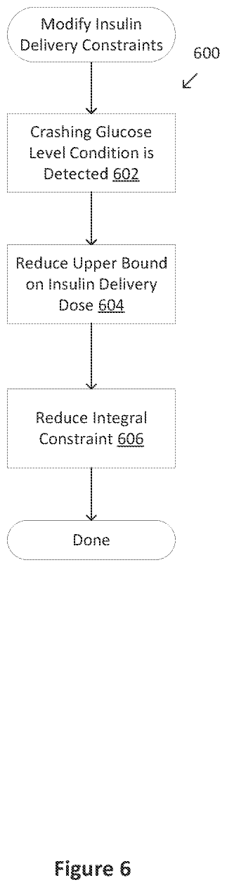

The response of the system to detecting a crashing glucose level condition may not be limited simply to modifying coefficients of the glucose level prediction model. Additional measures also may be taken. For instance, certain constraints may be modified responsive to detecting the crashing glucose level condition. FIG. 6 depicts a flowchart 600 of illustrative steps that may be performed in exemplary embodiments regarding modifying insulin delivery constraints. At 602, a crashing glucose level condition may be detected. In response, at 604, an upper bound on a one-time insulin delivery dose may be reduced. For example, the maximum basal insulin delivery dose may be reduced to be set at a value, such as two times the standard basal delivery dose. Additionally or alternatively, at 606, an integral constraint may be reduced. The integral constraint may limit how much insulin may be delivered by the medicament delivery device 102 over a period, such as a period of hours. For example, the limit may be reduced to 9 times the normal basal hourly delivery amount.

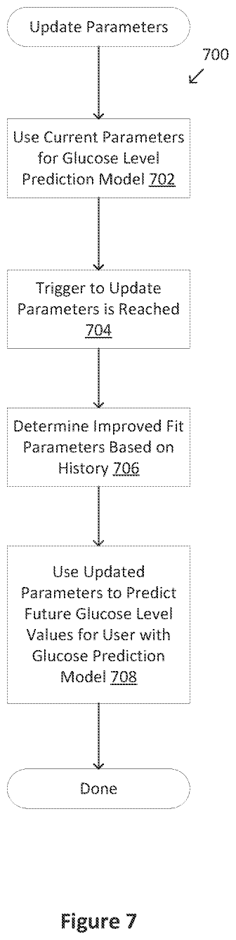

As was mentioned above, the parameters for the glucose prediction model may be customized for the user and updated to account for recent glucose level values. FIG. 7 depicts a flowchart 700 of steps that may be performed in exemplary embodiments in customizing the parameters of the glucose prediction model for the user 108. Initially, at 702, the current parameters are used by the glucose prediction model. At 704, a trigger to update the parameters is reached, such as reaching a new operational cycle, a new delivery cycle, a new hour, a new day, a new week, use or application of a new medicament delivery device in place of an old one, or other time period. The trigger may be customizable. A “run” is used herein to refer to the time period or cycle during which a glucose level value is received from the time of the last update and the last trigger point. More generally, an event or other trigger prompting an update of the parameters may be reached. At 706, parameters providing an improved fit relative to the glucose level values in the run may be determined. In some embodiments, the parameters providing the best fit to the user (or the received glucose level values) may be determined. At 708, the parameters with the improved fit are used to adapt the current parameters of the glucose prediction model in view of the glucose level or levels of the run, such as described below. At 710, the adapted parameters are applied in predicting a future glucose level value with the glucose prediction model.

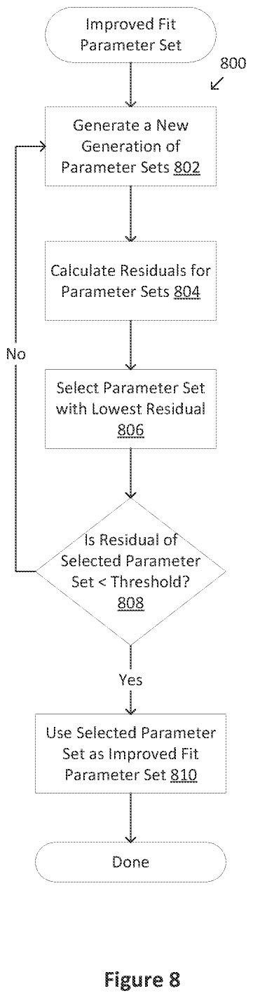

FIG. 8 depicts a flowchart 800 depicting illustrative steps that may be performed in an illustrative approach to generate an improved fit parameter set in accordance with exemplary embodiments. This approach uses a genetic algorithm that generates generations of parameter sets and mutates the generations (such as by perturbing values from the previous generation) until an improved fit parameter set (such as a best fit parameter set) is found. At 802, a new generation of parameter sets is generated. The initial parameter set may be chosen randomly and then a subsequent generation may be generated by mutating the best parameter set of the current generation. For the generation, at 804, residuals may be calculated for each parameter set in the generation. The residuals may be calculated using a fitness function. In this instance, the fitness function may calculate the differences between predicted parameters in each parameter set relative to the actual historical glucose level values. These differences may be aggregated for each parameter set to determine the aggregate residuals for the parameter set.

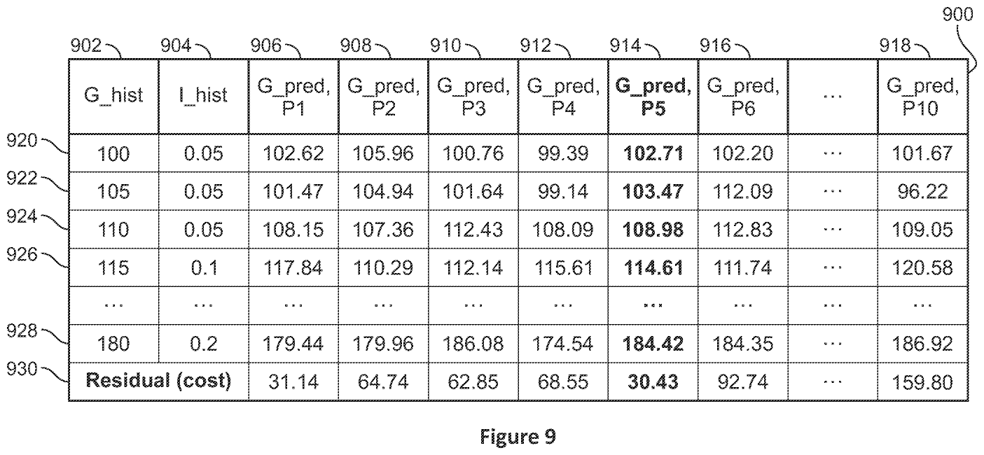

FIG. 9 depicts a table 900 of illustrative values for a generation of parameter set. Table 900 includes column 902 of historical glucose level values, and column 904 of historical insulin delivery amounts for the cycle used in the glucose prediction model. Columns 906, 908, 910, 912, 914, 916 and 918 are associated with predicted glucose level values generated by respective parameter sets designated as P1, P2, . . . , P10. Rows 920, 922, 924, 926, and 928 hold information regarding the predictions generated for an actual historical glucose level of the user and an insulin delivery amount for cycle k−3 as used in the glucose prediction model (see Equation 1). Thus, for row 920, the columns 906, 908, 910, 912, 914, 916 and 918 hold the predicted glucose values generated when the respective parameter sets are used with the predictive glucose model for cycle where the actual glucose level value was 100 mg/dL and an insulin delivery amount of 0.5 units. Row 930 holds the aggregate residuals for the respective parameter sets. As can be seen, parameter set P5 has the smallest residual. Thus, parameter set for P5 has the best fit for this generation.

With reference to FIG. 8, at 806, the parameter set with the smallest residual may be chosen. At 808, the residual for the selected parameter set may be compared to a threshold to determine if the parameter set is acceptable as the improved fit parameter set. If not, at 802, a new generation is produced by mutating the selected parameter set. If so, at 810, the selected parameter set may be used as the improved fit parameter set.

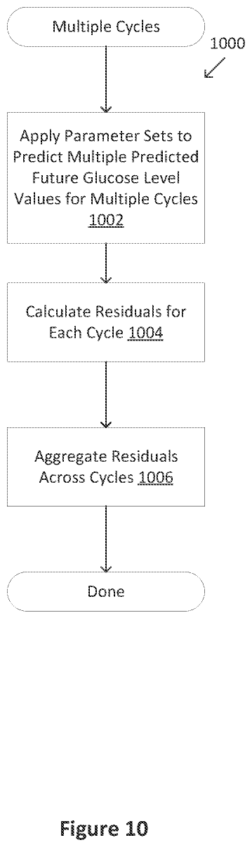

The adaptation need not seek to fit parameters for a single cycle but rather may seek to find the parameters across multiple cycles. FIG. 10 depicts a flowchart 1000 of illustrative steps that may be performed in exemplary embodiments to make predictions across multiple cycles. In such an instance, at 1002, parameter sets are applied to the glucose prediction model to predict future glucose level values of the user across multiple cycles. For example, glucose level values may be predicted for the next 12 cycles. At 1004, the predicted glucose level values are compared with the corresponding actual glucose level values for the cycles to calculate residuals for each cycle. At 1006, the residuals are aggregated across the cycles (i.e., summed). These aggregate residuals are then used in selecting the improved fit set of parameters as discussed above.

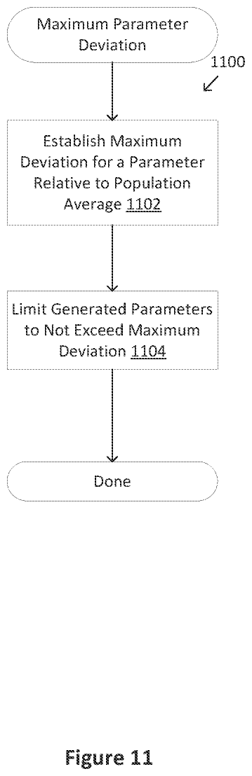

The parameters that may be created during the mutation phase to create a next generation may be constrained. FIG. 11 depicts a flowchart 1100 of illustrative steps that may be performed in exemplary embodiments to constrain such parameters. At 1102, a maximum deviation for a parameter relative to a population average (e.g., the average value of the parameter over the run) may be established. For instance, a parameter may not deviate from the average by more than a threshold amount, such as by more than 25%. At 1104, when parameter sets are generated via the process of finding an improved parameter fit, the parameters may not deviate from the average parameter value by more than the threshold amount.

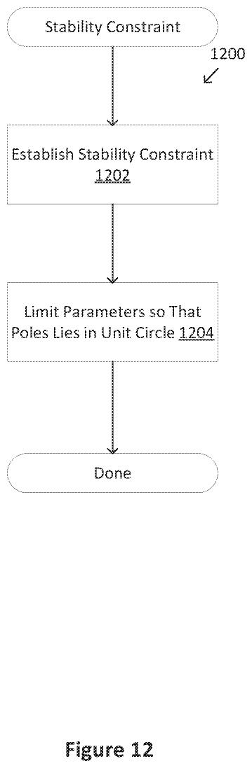

A stability constraint may also be applied to parameters. FIG. 12 depicts a flowchart of illustrative steps that may be applied in exemplary embodiments relating to the stability constraint. At 1202, a stability constraint is established. As a result, at 1204, parameters for the glucose prediction model are limited so that poles for the model lie in a unit circle. In other words solutions to the equation 1 b_{1,new}z⁻¹−b_{2,new}z⁻²−b_{3,new}z⁻³=0 must not exceed −1 to 1, where b_{i,new} represents a generated ith coefficient and z⁻¹ is an inverse z transform.

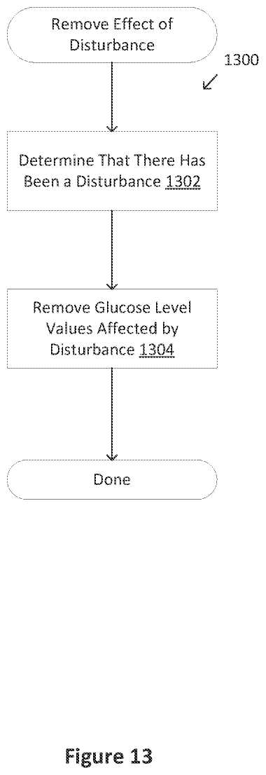

In order to avoid data that results from an external disturbance, glucose level values resulting from a disturbance, such as meal ingestion or exercise, may be removed from the data in a run that is processed to find the improved fit parameter set. FIG. 13 depicts a flowchart 1300 of illustrative steps that may be performed in exemplary embodiments to remove the effect of such a disturbance. At 1302, a determination may be made that there has been a disturbance. For instance, the user may have indicated that they have ingested a meal or are exercising. Alternatively, the medicament delivery system of FIG. 1 may include logic in software for processing glucose level values and making the determination that there is a disturbance. For example, glucose level values may increase rapidly indicating meal ingestion or may decrease in a manner indicative of exercise. At 1304, glucose level values affected by the disturbance may be removed from the run that is processed in updating the parameter set to the improved fit parameter set. For example, glucose level values following a meal may be removed, such as by omitting glucose level values for a period of time, such as a period of 5 hours, following meal ingestion of a meal of a size over a threshold, like a meal having at least 40 grams of carbohydrates. Similarly, glucose level values for an exercise period may be omitted.

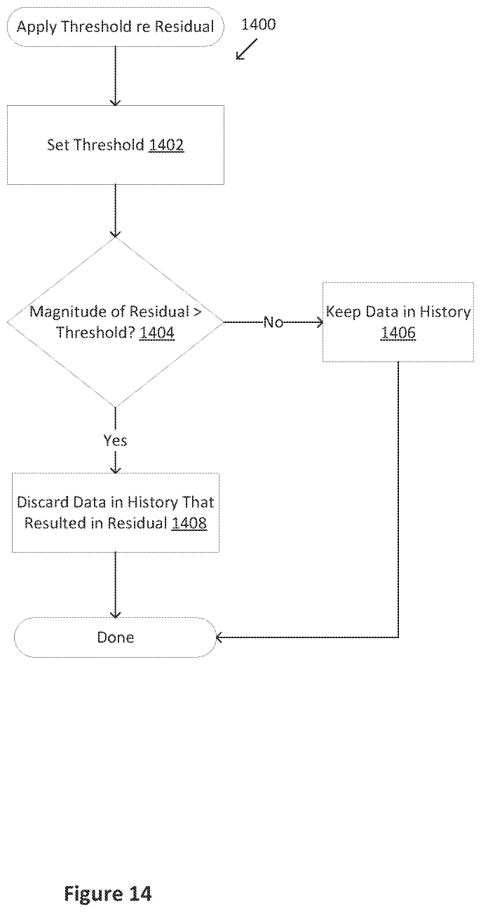

Another approach to limiting the effect of the disturbances is depicted in the flowchart 1400 of FIG. 14. FIG. 14 depicts a flowchart 1400 of illustrative steps that may be performed in exemplary embodiments to remove glucose level values affected by a disturbance. At 1402, a threshold may be established. The threshold may represent how much a residual may vary from a threshold value. The threshold may be a percentage of the actual glucose level value. For example, a threshold may establish that residuals indicative of the actual glucose level value varying by more than 20% from the predicted glucose level value may be discarded or not considered. Other percentages or absolute values (e.g., more than 20 mg/dL) may be used. Alternatively, the threshold may be based on average residual values and may represent a maximum permitted deviation in a residual value relative to the average residual value (e.g., one standard deviation). At 1404, the magnitude of the residual between a glucose level prediction and a predicted glucose level value may be compared to the threshold. If the residual exceeds the threshold, at 1408, the actual glucose level value may be removed from the glucose level history. Otherwise, at 1406, the glucose level value may be kept in the glucose level history.

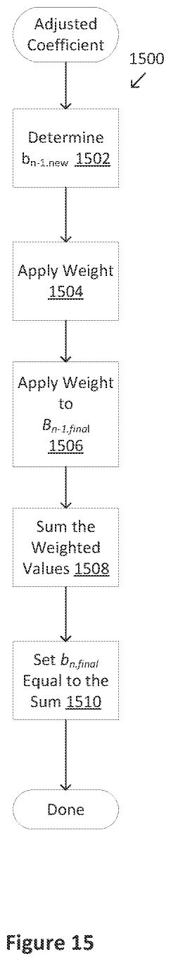

Once the improved fit parameters have been determined based on the newly processed run of glucose level values the glucose level prediction model may be adapted to reflect the improved for parameters more. FIG. 15 depicts a flowchart 1500 of illustrative steps that may be performed to adapt the parameters. In a specific example, each coefficient b₁, b₂, and b₃, or other parameters, may be adapted to reflect changes in the improved fit parameter values relative to the previous parameter values, and this may be done for each “run” or each cycle, as explained above with reference to FIG. 7. Continuing with the b coefficient example, at 1502, a value may be determined for b_{n-1,new}, which is the value for the n−1 cycle for the coefficient in the improved fit parameter set, such as described above. At 1504, a weight may be applied to b_{n-1,new}. For example, the weight may be 0.8 times the number of days in the run N_{days,new}. At 1506, a weight may be applied to the coefficient weight used to predict glucose values for cycle n−1 using the current parameter set. A suitable weight may be 0.2 times the number of days in the run. In this manner, using these exemplary numbers, if there is only one new day of data, then the prior b coefficient (b_{n-1, final}) would be weighted 80% when determining a new b coefficient (b_{n, final}) Other weightings may be used. At 1508, these weighted values may be summed. At 1510, the new coefficient value, b_{n, final} may then be set equal to the sum. This sum may be expressed as:

b_{n,final}=(1−0.2·N_{days,new})b_{n-1,final}+(0.2·N_{days,new})b_{n-1,new} (Equation 6).

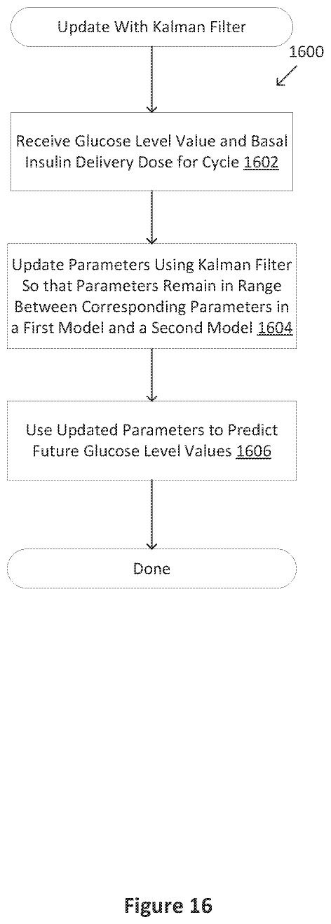

Adaptation of parameter values may be used in cases where multiple models are used, such as described above. It is assumed that there are at least two glucose prediction models and which glucose prediction model depends on current conditions. FIG. 16 depicts a flowchart 1600 of illustrative steps that may be performed in exemplary embodiments to adapt the parameters on an ongoing basis using a Kalman filter. The Kalman filter may use the inputs and statistical noise to produce the estimates by estimating the joint probability distribution over the parameters for each cycle. At 1602, a most recent glucose level value for the user and a basal insulin dose that was most recently delivered may be received. In response to these inputs, at 1604, parameters for the glucose prediction model may be updated using a Kalman filter, for example. At 1606, the updated parameters may then be applied to the glucose prediction model to estimate one or more glucose level values for the user.

While exemplary embodiments have been described herein, various changes in form and detail may be made without departing from the intended scope of the appended claims.

1. A medicament delivery system for delivering medicament to a user, comprising:
a non-transitory computer-readable storage medium storing computer programming instructions;
a processor configured for executing the computer programming instructions to cause the processor to:
predict a first future glucose level of the user using a first model that predicts the future glucose level based on glucose level history of the user and medicament deliveries to the user;
detect a crashing glucose level condition of the user;
in response to the detecting of the crashing glucose level condition, switching to a second model to predict a next future glucose level of the user, the second model having different parameter values than the first model such that the second model predicts lower future glucose levels for the user than the first model when glucose levels of the user are decreasing.

2. The medicament delivery system of claim 1, wherein the processor detects the crashing glucose level condition by determining differences between successive pairs of glucose level readings of a sequence of most recent glucose level readings of the user that extends from an oldest glucose level reading in the sequence to a most recent glucose level reading in the sequence and comparing each of the differences to a threshold.

3. The medicament delivery system of claim 2, wherein the processor detects the crashing glucose level condition by comparing the differences to each other to determine if the differences between the successive pairs of glucose level readings of the user become more negative as the differences range from an oldest glucose level reading pair to a most recent glucose level reading pair.

4. The medicament delivery system of claim 3, wherein the processor detects a crashing glucose level condition of the user when each of the differences is more negative than the threshold, each of the differences is negative, and the differences between the successive pairs of glucose level readings of the user become more negative as the differences range from the oldest glucose level reading pair to the most recent glucose level reading pair.

5. The medicament delivery system of claim 4, wherein the processor detecting a crashing glucose level condition also entails comparing Insulin on Board (IOB) of the user to a threshold.

6. The medicament delivery system of claim 1, wherein the processor detects the crashing glucose level condition by looking for glucose level readings of the user dropping at an accelerating rate.

7. The medicament delivery system of claim 1, wherein in response to the detecting of the crashing glucose level condition, the processor decreases an upper bound of an amount of insulin that may be delivered by the medicament delivery device to the user over a time period.

8. The medicament delivery system of claim 1, wherein the computer programming instructions when executed further cause the processor to determine a basal insulin delivery dose for the user based on the predicted next future glucose level of the user that was predicted using the second model.

9. The medicament delivery system of claim 8, further comprising a cannula and/or needle for delivering the medicament to the user and wherein the computer programming instructions when executed by the processor further cause the processor to initiate delivery of the basal insulin delivery dose to the user via the needle and/or cannula.

10. A medicament delivery system for delivering medicament to a user, comprising:
a non-transitory computer-readable storage medium storing computer programming instructions;
a processor configured for executing the computer programming instructions to cause the processor to:
access a history of medicament delivery doses and glucose level values of the user;
based on the history, determine an improved fit of parameters for a glucose prediction model from the history relative to current parameters of the glucose prediction model, wherein the glucose prediction model determines predicted future glucose level values for the user from past glucose level values and past medicament delivery doses in the history, such that glucose prediction model using the improved fit parameters more accurately predicts future glucose level values than the glucose prediction model using the current parameters to predict future glucose level values when compared to predicting glucose level values in the history of glucose level values of the user;
adapting the parameters based on the improved fit of parameters to produce adapted parameters; and
use the glucose prediction model with the adapted parameters to predict at least one future glucose level value of the user.

11. The medicament delivery system of claim 10, in determining the improved fit of parameters for the glucose prediction model, the glucose prediction model determines a best fit of parameters for the glucose prediction model.

12. The medicament delivery system of claim 11, wherein the improved fit is the best fit of parameters for the glucose prediction model.

13. The medicament delivery system of claim 10, wherein the processor uses a genetic algorithm to determine the improved fit of the parameters.

14. The medicament delivery system of claim 10, wherein the medicament delivery device has operational cycles, receives a glucose level reading each operational cycle, and delivers a basal insulin dose to the user each operational cycle, and wherein the processor in determining the improved for of parameters examines over multiple operational cycles of the medicament delivery device.

15. The medicament delivery system of claim 10, wherein a maximum deviation of a parameter relative to a population average for parameter candidates is established and at least one of the parameters in the improved fit of parameters is subject to the maximum deviation.

16. The medicament delivery system of claim 10, wherein parameters in the improved fit of parameters must conform with a stability constraint.

17. The medicament delivery system of claim 10, wherein glucose level values in the history of glucose level values that are affected by disturbances are removed from consideration in determining the improved fit of parameters.

18. A medicament delivery system for delivering medicament to a user, comprising:
a non-transitory computer-readable storage medium storing computer programming instructions;
a processor configured for executing the computer programming instructions to cause the processor to:
use a current model to predict a response in analyte levels of the user to delivering a dose of medicament;
responsive to detecting a condition, swap the current model for an additional model or updating the current model based on new analyte level data; and
use the additional model or the updated current model to modify medicament dose delivery for the user.

19. The medicament delivery system of claim 18, wherein the detected condition is rapidly changing analyte level data.

20. The medicament delivery system of claim 18, wherein the modification is to halt delivery of the medicament.
